(19) United States
(12) Patent Application Publication
(10) Pub. No.: US 2012/0052008 A1
(43) Pub. Date: Mar. 1, 2012

(54) NANOSTRUCTURES COMPRISING RADIOISOTOPES AND/OR METALS

(76) Inventors: Yuri Mackeyev, Houston, TX (US); Lon J. Wilson, Houston, TX (US); Izabela Tworowska, Houston, TX (US)

(21) Appl. No.: 13/218,213

(22) Filed: Aug. 25, 2011

Related U.S. Application Data
(60) Provisional application No. 61/376,972, filed on Aug. 25, 2010.

(51) Int. Cl. A61K 51/12; A61P 43/00; A61K 51/02; B82Y 5/00

(52) U.S. Cl. 424/1.61; 977/734; 977/773; 977/927

(57) ABSTRACT

Nanostructures comprising radioisotopes and/or metals are provided. More particularly, in some embodiments, nanostructures comprising radioisotopes and/or metals, methods of their synthesis, and their use in cancer imaging and therapy are provided.

### CROSS REFERENCE TO RELATED APPLICATIONS

This application claims the benefit of U.S. Provisional Application No. 61/376,972, filed Aug. 25, 2010, the entire disclosure of which is hereby incorporated by reference.

### BACKGROUND

Since their discovery in 1991, carbon nanotubes have found wide-spread potential for various technological applications. In particular, their hollow interior coupled with a chemically-modifiable outer surface makes them intriguing candidates as diagnostic and therapeutic agents in medicine. Single-walled carbon nanotubes (SWNTs), which can be described as hollow cylinders made from single sheets of graphene, are among the most investigated form of carbon nanotubes for biological and medical applications. The ideal SWNT length for biological applications is still unknown, however, ultra-short SWNTs (US-tubes), 20-100 nm in length, might be especially good candidates for such applications.

### SUMMARY

The present disclosure relates generally to nanostructures comprising radioisotopes and/or metals. More particularly, in some embodiments, the present disclosure relates to nanostructures comprising radioisotopes and/or metals, methods of their synthesis, and their use in cancer imaging and therapy.

In one embodiment, the present disclosure provides a composition comprising a Cₙ carrier, wherein Cₙ refers to a fullerene moiety or nanotube comprising n carbon atoms, and at least one radioisotope.

In another embodiment, the present disclosure provides a composition comprising: a Cₙ carrier, wherein Cₙ refers to a fullerene moiety or nanotube comprising n carbon atoms, and at least one agent selected from the group consisting of a radioisotope, a metal and a combination thereof.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 is a graph depicting the results of co-loading a Cₙ carrier (e.g., carbon nanotube) with Lu¹⁷⁷ chloride and GdCl₃. The graph shows the correlation between activity of Lu¹⁷⁷ loaded in nanotubes (that is defined as radiochemical yield of absorption—RCY_{Ab}) and pH of buffer used for loading (0.1M NaOAc pH=6.1, 0.1M HEPES pH=8.2). Radiochemical yield is not back-decayed.

FIG. 2 is a graph depicting the results of co-loading a Cₙ carrier with Lu¹⁷⁷ chloride and Gd chloride. The graph shows the correlation between activity of Lu¹⁷⁷ loaded in nanotubes (defined as radiochemical yield of desorption—RCY_{Ds}) and pH of the loading reaction. Loaded nanotubes were washed out with ddH2O (Ultra pure, 3×1 mL), PBS 1× (3×1 ml), 1 mM EDTA pH=4.3 (3×1 mL), 1 mM EDTA pH=7.3 (3×1 mL), 1 mM EDTA pH=8.3 (3×1 mL). Calculated RCY is not-decay-corrected.

FIG. 3 is a graph depicting the results of stability tests of a Cₙ carrier loaded with Lu¹⁷⁷ and GdCl₃ in 0.1M NaOAc pH=6.3 and 0.1M HEPES pH=8.3. The graph shows the percentage of radioactivity remained in nanotubes after washing them with 3 mL of PBS buffer (pH=7.2). Calculated radioactivity is not-decay corrected.

FIG. 4 is a graph depicting the efficacy of loading a Cₙ carrier with cold LuCl₃ and GdCl₃ performed in buffers at pH 5.2, 6.4 and 7.4, respectively. The weight percentages of co-loading both metals in nanotubes were determined by ICP-MS.

FIG. 5 is a graph depicting the efficacy of loading a Cₙ carrier with both Lu¹⁷⁷ and Gd or Lu¹⁷⁷ only and it is described by radiochemical yield of absorption. Both reactions (loading nanotubes with Lu¹⁷⁷ and Gd or Lu¹⁷⁷ alone) were done in the same buffer conditions (0.1M HEPES pH=8.3).

FIG. 6 is a graph depicting the results of the challenge studies of Lu¹⁷⁷-loaded carbon nanotubes. The graph shows the activity of Lu¹⁷⁷ in nanotubes (RCY_{Ds}) after washing them with 1 mM EDTA pH=4.3, 7.3 or 8.3, respectively.

FIG. 7 is a graph depicting the results of loading a Cₙ carrier with ^{99m}TcO₄ and GdCl₃ (^{99mTcO4}/Gd for short) in the presence of SnCl₂. The graph shows RCY_{Ab} of ^{99m}TcO₄ and Gd loaded in 0.1M NaOAc buffer at pH=6.1 and 0.1M HEPES at pH=8.3, respectively.

FIG. 8 is a graph depicting the RCY_{DS} of loading a Cₙ carrier with ^{99m}TcO₄ and Gd chloride in 0.1M NaOAc pH=6.1 and 0.1M HEPES pH=8.3, respectively and washing them with 1 mM EDTA.

FIG. 9 is a graph depicting the results of loading a Cₙ carrier with ⁶⁸Ga and Gd in 0.1M NaOAc and washing it with 0.1M EDTA pH=4.3 and 1 mM EDTA pH=4.3. The graph shows the weight percentages of Gd remained in the carriers after washing them with EDTA as were determined by ICP-MS.

FIG. 10 is a graph depicting the RCY_{Ab} of loading a Cₙ carrier with ⁶⁸Ga/Gd in 0.1M NaOAc pH=4.1 and 0.1M NaOAc pH=6.3. Calculated RCY was back-decayed.

FIG. 11 is a graph depicting the RCY_{D}, of loading a Cₙ carrier with ⁶⁸Ga/Gd in 0.1M NaOAc pH=4.1 and 0.1M NaOAc pH=6.3. Calculated RCY was back-decayed.

FIG. 12 is a graph depicting the stability of loading a Cₙ carrier with ^{99m}TcO₄ in the presence of SnCl₂ after washing them with PBS buffer (1×, 3 mL) and) and 0.1M EDTA pH=7.6-9.1.

FIG. 13 is a graph depicting the efficacy of loading a Cₙ carrier with Re compounds (1) ReCl₅, (2) (NBu)₄ReClO₄— and (3) NH₄ReO₄ as determined by ICP-MS.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure relates generally to nanostructures comprising radioisotopes and/or metals. More particularly, in some embodiments, the present disclosure relates to nanostructures comprising radioisotopes and/or metals, methods of their synthesis, and their use in cancer imaging and therapy.

In some embodiments, the compositions of the present disclosure may be used as multimodality probes for detection of disease-changed organs using different detection methods. These novel probes will allow for acquisition of different functional parameters with PET and SPECT techniques in addition to high-resolution anatomical and functional information that can be obtained with MRI and CT techniques. The compositions of the present disclosure can enhance the diagnostic information that can be acquired in a single imaging session. Compositions of the present disclosure can be detected by one or more imaging methods. In some embodiments, the compositions of the present disclosure may be used as dual modality PET/CT, PET/MRI agents or multimodality PET/CT/MRI/SPECT agents.

In addition to imaging functions, compositions of the present disclosure may also be used in therapy. Compositions of the present disclosure may be used as single agents and can be detected by hybrid PET/CT camera saving patients from multiple injections. The same multimodal functions of these compositions may also apply for MRI/PET scanners.

Of the many potential advantages of the present disclosure, many of which are not discussed herein, the compositions of the present disclosure can be used to enhance sensitivity of existing imaging techniques and promote development of new multimodality methods. These compositions can be potentially used in radiation dosimetry to determine actual dose and absorbed dose of activity that has to be delivered to the target tissues. They can be also functionalized with tumor targeting ligands and be used to evaluate their efficacy. They can also be used as imaging agents to assess treatment response to the therapy.

In some embodiments, the compositions of the present disclosure may act as a replacement for chelates in medicine and mainly nuclear medicine. In this way, various combinations of metal ions (and other medical agents like I₂ or cis-platin) can be simultaneously loaded into the compositions to produce multimodal imaging and therapeutic agents in any desired combination.

In one embodiment, the present disclosure provides a composition comprising: a Cₙ carrier, wherein Cₙ refers to a fullerene moiety or nanotube comprising n carbon atoms, and at least one selected from the group consisting of a radioisotope, a metal and a combination thereof. As used herein, a Cₙ carrier refers to a fullerene moiety comprising n carbon atoms or a nanotube moiety comprising at least n carbon atoms.

Examples of suitable Cₙ carriers for use in conjunction with the compositions of the present disclosure include, but are not limited to, buckminsterfullerenes, gadofullerenes, single walled carbon nanotubes (SWNTs), and ultra-short carbon nanotubes (US-tubes). Buckminsterfullerenes, also known as fullerenes or more colloquially, buckyballs, are closed-cage molecules consisting essentially of sp²-hybridized carbons. Fullerenes are the third form of pure carbon, in addition to diamond and graphite. Typically, fullerenes are arranged in hexagons, pentagons, or both. Most known fullerenes have 12 pentagons and varying numbers of hexagons, depending on the size of the molecule. Common fullerenes include C₆₀ and C₇₀ (e.g. n=60 or n=70), although fullerenes comprising up to about 400 carbon atoms are also known.

SWNTs, also known as single walled tubular fullerenes, are cylindrical molecules consisting essentially of sp² hybridized carbons. In defining the size and conformation of single-walled carbon nanotubes, the system of nomenclature described by Dresselhaus et al., Science of Fullerenes and Carbon Nanotubes, Ch. 19 will be used. Single walled tubular fullerenes are distinguished from each other by a double index (x,y), where x and y are integers that describe how to cut a single strip of hexagonal graphite such that its edges join seamlessly when the strip is wrapped onto the surface of a cylinder. When x=y, the resultant tube is said to be of the “arm-chair” or (x,x) type, since when the tube is cut perpendicularly to the tube axis, only the sides of the hexagons are exposed and their pattern around the periphery of the tube edge resembles the arm and seat of an arm chair repeated n times. When y=0, the resultant tube is said to be of the “zig-zag” or (x,0) type, since when the tube is cut perpendicular to the tube axis, the edge is a zig zag pattern. Where x≠y and y≠0, the resulting tube has chirality. The electronic properties of the nanotube are dependent on the conformation, for example, arm-chair tubes are metallic and have extremely high electrical conductivity. Other tube types are metallic, semi-metals, or semi-conductors, depending on their conformation. Regardless of tube type, all SWNTs have extremely high thermal conductivity and tensile strength. The SWNT may be a cylinder with two open ends, a cylinder with one closed end, or a cylinder with two closed ends. Generally, an end of an SWNT can be closed by a hemifullerene, e.g. a (10,10) carbon nanotube can be closed by a 30-carbon hemifullerene. If the SWNT has one or two open ends, the open ends can have any valences unfilled by carbon-carbon bonds within the single wall carbon nanotube filled by bonds with hydrogen, hydroxyl groups, carboxyl groups, or other groups. SWNTs can also be cut into ultra-short pieces, thereby forming US-tubes. As used herein, the term “US-tubes” refers to ultra short carbon nanotubes with lengths from about 20 nm to about 100 nm.

The Cₙ carriers useful in the compositions and methods of the present disclosure may be produced by any means known to one of ordinary skill in the art. In certain embodiments, the Cₙ carriers useful in the compositions and methods of the present invention may be produced by electric arc discharge. In certain embodiments, the Cₙ carriers useful in the compositions and methods of the present invention may be produced by high pressure CO conversion (HiP_{CO}). A substantial amount of previous research concerning the loading of SWNT samples has been performed with electric-arc discharge-produced SWNTs as opposed to other SWNT production methods, such as high-pressure carbon monoxide (HiP_{CO}). This is because, in many cases, arc-produced SWNTs have, among other things, a larger diameter than HiP_{CO} SWNTs (1.4 nm average diameter for arc vs. 1.0 nm diameter for HiP_{CO}) and arc SWNTs may contain more sidewall defects than HiP_{CO} SWNTs, thereby facilitating loading. For medical applications, however, the uniformity and purity of HiP_{CO} SWNTs may be advantageous. Suitable commercially available carbon nanotubes may be obtained from Carbon Nanotechnologies Inc., Houston, Tex.

In certain embodiments, methods of producing US tubes may comprise cutting full-length SWNTs into short pieces by a four-step process. First, residual iron catalyst particles may be removed by oxidation via exposure to wet-air or SF₆ followed by a strong acid (HCl) treatment to extract the oxidized iron particles. The purified SWNTs may then be fluorinated by a gaseous mixture of 1% F₂ in He at elevated temperatures for up to 2 hours and cut into short pieces by pyrolysis under argon at 900° C. The fluorination reaction may produce F-SWNTs, with a stoichiometry of CFₓ (x<0.2), which may comprise bands of fluorinated-SWNT separated by regions of pristine SWNT. Pyrolysis under argon, among other things, liberates volatile fluorocarbons, thereby cutting the SWNTs into pieces with lengths corresponding to the areas of pristine SWNT. While this method known in the art is effective at producing cut SWNTs, improvements can be made; for example, the separate purification step is unnecessary and can be eliminated. Such improvements, provided that they do not adversely affect the compositions and methods of the present invention, are considered within this spirit of the present disclosure.

In certain embodiments, a three-step process of producing US tubes may be used. First, as produced HiP_{CO} SWNTs may be fluorinated in a monel steel apparatus by a mixture of 1% F₂ in He at 100° C. for about 2 hours. During this process, both the SWNTs and the iron catalyst particles may become at least partially fluorinated. Subsequent exposure to concentrated HCl may substantially remove the fluorinated catalyst particles without affecting the F-SWNTs, which have a stoichiometry of ˜C₁₀F after the acid treatment. The now-purified F-SWNTs are cut into US tubes by pyrolysis under argon at 900° C.-1000° C. In certain embodiments, the resulting US tubes have lengths ranging from 20-80 nm, with the majority being ˜40 nm in length. Utilizing this method, the amount of iron catalyst may be reduced from ˜25 mass percent in raw SWNTs to ˜1 mass percent for US tubes. Therefore, in certain embodiments, this method may be ideal for the purification of SWNTs, but only as a precursor to producing US tubes. This is because the fluorine remaining, after the HCl acid treatment, is difficult to remove, making the F-SWNTs only viable for subsequent cutting. Furthermore, the time to produce US tubes from SWNTs using this method may be significantly reduced.

A Cₙ carrier suitable for use in the present disclosure can be substituted or unsubstituted. By “substituted” it is meant that a group of one or more atoms is covalently linked to one or more atoms of a Cₙ carrier. Generally, in situ Bingel chemistry may be used to substitute a Cₙ carrier with appropriate groups to form a targeted nanostructure. Examples of groups suitable for use include, but are not limited to, malonate groups, serinol malonates, groups derived from malonates, serinol groups, carboxylic acid, polyethyleneglycol (PEG), and the like. In one embodiment, a Cₙ carrier is substituted with one or more water-solubilizing groups. Water-solubilizing groups are polar groups (that is, groups having a net dipole moment) that render the generally hydrophobic fullerene core soluble in water. The addition of such groups allow for greater biocompatibility of a Cₙ carrier. Generally, a Cₙ carrier may contain from 1 to 4 addends. A Cₙ carrier can be substituted with any water solubilizing group to allow for sufficient water solubility and biocompatibility, but the spectroscopic properties of the Cₙ carrier should not be compromised. In certain embodiments, a Cₙ carrier may be further substituted with either a thiol (—SH) or an amine (—NH₂) group.

As mentioned above, compositions of the present disclosure further comprise a radioisotope, a metal or a combination thereof. Examples of a suitable radioisotopes may include, but are not limited to, isotopes of technetium (e.g., ^{99m}Tc), gallium (e.g., ⁶⁸Ga), lutetium (e.g., ¹⁷⁷Lu), indium (e.g., ¹¹¹In), actinium (e.g., ²²⁷Ac, ²²⁵Ac) and their combination with Gd³⁺. In some embodiments, the compositions of the present disclosure may comprise metal ions such as gadolinium (e.g., Gd³⁺), rhenium (e.g., ¹⁸⁸Re), etc.

In some embodiments, a Cₙ carrier may be loaded with an isotope or an isotope in combination with Gd³⁺ by adding the isotope and/or metal to a Cₙ carrier suspended in buffer (pH=4.8-8.3) and sonicating for approximately one hour at 60° C.-70° C. Radiochemical yield and stability of loading the Cₙ carrier may be determined by washing the carriers with ddH2O (Ultra pure), phosphate buffer (PBS 1×), and EDTA (1 mM) at pH=4.3, pH=7.3, and pH=8.3. Absorption of GdCl₃ in nanotubes may be determined by ICP-MS.

In some embodiments, the compositions of the present disclosure can be used directly as MRI, PET, SPECT agents but can be potentially modified with tumor-targeting ligands. In some embodiments, functionalization of the compositions occurs after they have been loaded with a metal and/or radioisotope.

In certain specific embodiments, a composition of the present disclosure may comprise ^{99m}Tc, ¹⁷⁷Lu, ⁶⁷Ga and/or ¹¹¹In for use in SPECT imaging, ⁶⁸Ga for use in PET imaging, Gd³⁺ for use in MRI, ¹⁸⁸Re, ¹⁸⁶Re ²²⁷Ac and/or ²²⁵Ac for use as therapeutic agents, and any combination thereof.

Other suitable materials may be added to the compositions of the disclosure. For example, the presence of the hollow interior of the Cₙ carrier may allow materials including, but not limited to, multi-modal imaging agents and drugs to be administered by being contained substantially within the interior of the Cₙ carrier. The exterior wall of Cₙ carrier may also allow for the attachment of multi-modal imaging agents, targeting agents (including, but not limited to, peptides and antibodies) and/or additional therapeutic agents (including, but not limited to, chemotherapeutic agents and radiotherapeutic agents).

To facilitate a better understanding of the present invention, the following examples of certain aspects of some embodiments are given. In no way should the following examples be read to limit, or define, the entire scope of the invention.

### Example 1

HiP_{CO} single-wall nanotubes (SWNTs) were chemically cut into US-tubes, sonicated at RT in 0.5M HNO₃ for 30 minutes-2 hours. US-tubes were loaded with radioisotopes: ¹⁷⁷LuCl₃, ^{99m}TcO₄⁻, ⁶⁸GaCl₃, and cold Re compounds (ReCl₅, NH₄ReO₄, (NBu₄)[ReOCl₄]) manually or using automated module. The radiochemical yield was determined by radio-TLC and ICP-OES (Inductively coupled plasma optical emission spectrometry). Stability tests of labeled US-tubes were performed at RT for 1-24 h using 1M PBS, 0.1% FBS and transchelator (0.1M or 1 mM of EDTA) to determine the desorption half-life of the labeled US-tubes.

Ga⁶⁸-loading: Loading of US-tubes with ⁶⁸Ga was performed in 0.5M NH₄OAc buffer at 90° C. for 10 min using ⁶⁸GaCl₃ eluted from ⁶⁸Ge/⁶⁸Ga generator. Yield of the synthesis was found to be pH dependent with over 70% loading at pH=3.6-4.1 and to decrease drastically to 1% at pH<1.7.

^{99m}Tc-loading: Optimum ^{99m}Tc labeling of US-tubes proceeded in the presence of SnCl₂ (reducing agent, which turns pertechnetate into hydrated technetium dioxide, TcO₂.H₂O) in 0.5M NH₄OAc buffer with final yield >51%. Yield of the reaction decreased to 1% in the absence of SnCl₂, or when reduction proceeded after completion of labeling.

Lu¹⁷⁷-loading: Best ¹⁷⁷Lu loading of US-tubes was performed in 0.1M NH₄OAc at pH=5.1 at 90° C. for 20 min. The final yield of this synthesis was found to be >55% after repeated dialysis with 0.1M EDTA, pH=4.7.

Re-loading (cold metal): Optimum conditions for loading of US-tubes with cold Re compounds were achieved for ReCl₅ and (NBu₄)[ReOCl₄] applied as solutions to the nanotube material, preliminary treated with NaOH solution and then washed. Incubation was performed for 1 h at 95° C., after which the sample was washed and dried. The resulting US-tubes contain 17-23% of Re (by weight).

### Example 2

### Loading of Carbon Nanotubes with Lu¹⁷⁷ Chloride and Gd Chloride

US-tubes (100-200 ug) were sonicated for 1 h in 3-ml 0.5M HNO₃ (Ultra pure, trace metal free) in water bath at 60° C., and spin down at 10 k rpm for 15 minutes. Nanotubes were resuspended in HEPES buffer pH=8.3 and Lu¹⁷⁷ chloride (UM Research Reactor) and GdCl₃ (Sigma Aldrich) were added (final pH=8.0) to the solution. Suspension was sonicated for 30 minutes at 60° C. Stability of loading the nanotubes was determined by washing them through the Teflon filter with phosphate buffer (PBS 1×, 3 mL), 1 mM solution of EDTA pH=4.3, pH=7.3, and pH=8.3 (each 3 mL). Absorption of GdCl₃ in the nanotubes was determined by ICP-MS.

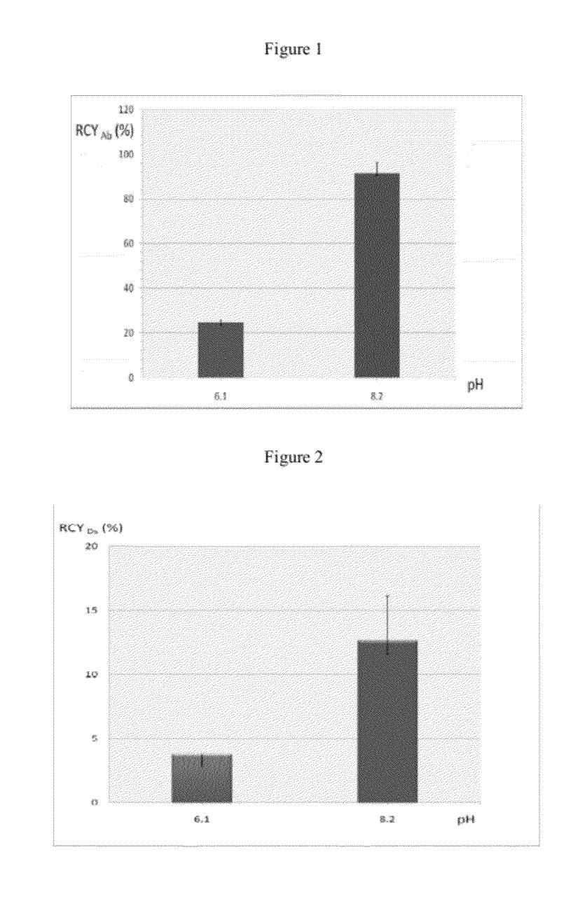

FIG. 1 is a graph depicting the results of co-loading the carbon nanotubes with Lu¹⁷⁷ chloride and GdCl₃. The graph shows the correlation between activity of Lu¹⁷⁷ loaded in nanotubes (that is defined as radiochemical yield of absorption—RCY_{Ab}) and pH of buffer used for loading (0.1M NaOAc pH=6.1, 0.1M HEPES pH=8.2). Radiochemical yield is not back-decayed.

FIG. 2 is a graph depicting the results of co-loading carbon nanotubes with Lu¹⁷⁷ chloride and Gd chloride. The graph shows the correlation between activity of Lu¹⁷⁷ loaded in nanotubes (defined as radiochemical yield of desorption—RCY_{Ds}) and pH of the loading reaction. Loaded nanotubes were washed out with ddH2O (Ultra pure, 3×1 mL), PBS 1× (3×1 ml), 1 mM EDTA pH=4.3 (3×1 mL), 1 mM EDTA pH=7.3 (3×1 mL), 1 mM EDTA pH=8.3 (3×1 mL). Calculated RCY is not-decay-corrected.

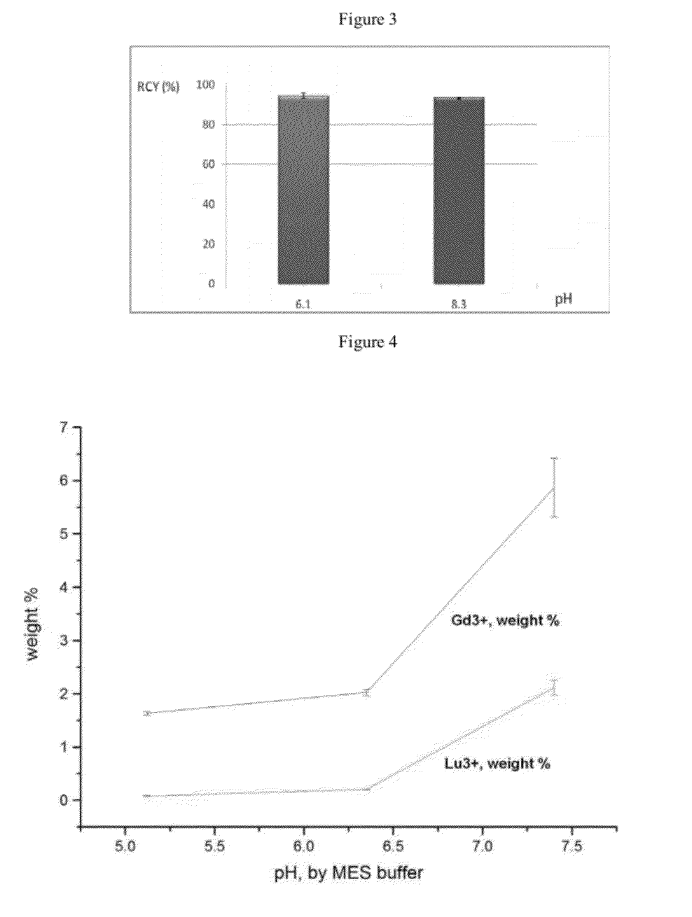

FIG. 3 is a graph depicting the results of stability tests of carbon nanotubes loaded with Lu¹⁷⁷ and GdCl₃ in 0.1 M NaOAc pH=6.3 and 0.1 M HEPES pH=8.3. The graph shows the percentage of radioactivity remained in nanotubes after washing them with 3 mL of PBS buffer (pH=7.2). Calculated radioactivity is not-decay corrected.

FIG. 4 is a graph depicting the efficacy of loading carbon nanotubes with cold LuCl₃ and GdCl₃ performed in buffers at pH 5.2, 6.4 and 7.4, respectively. The weight percentages of co-loading both metals in nanotubes were determined by ICP-MS.

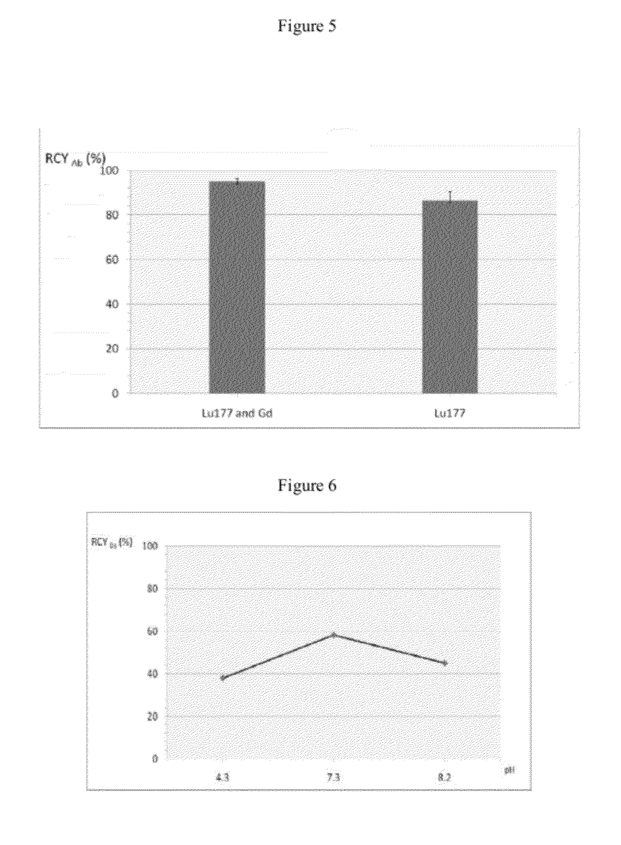

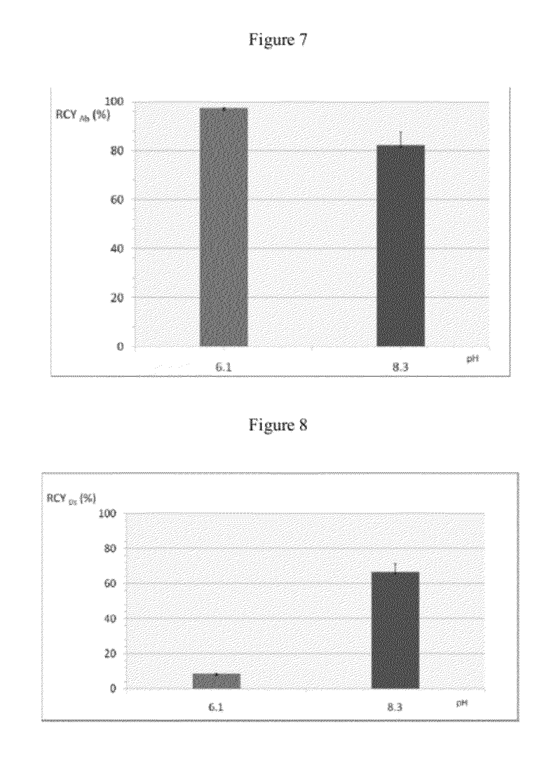

FIG. 5 is a graph depicting the efficacy of loading carbon nanotubes with both Lu¹⁷⁷ and Gd or Lu¹⁷⁷ only and it is described by radiochemical yield of absorption. Both reactions (loading nanotubes with Lu¹⁷⁷ and Gd or Lu¹⁷⁷ alone) were done in the same buffer conditions (0.1M HEPES pH=8.3).

FIG. 6 is a graph depicting the results of the challenge studies of Lu¹⁷⁷-loaded carbon nanotubes. The graph shows the activity of Lu¹⁷⁷ remained in nanotubes (RCY_{Ds}) after washing them with 1 mM EDTA pH=4.3, 7.3 or 8.3, respectively.

Loading of Carbon Nanotubes with ^{99m}TcO₄ and Gd Chloride

US-tubes (100-200 ug) were sonicated for 1 hour in 3-ml 0.5M HNO₃ (ultra pure, trace metal free) in water bath at 60° C. and spin down at 10 k rpm for 15 minutes. Nanotubes were resuspended in HEPES buffer pH=8.3 and ^{99m}TcO₄ (Triad Isotope Inc.), SnCl₃ (Sigma Aldrich) and GdCl₃ (Sigma Aldrich) were added (final pH=8.0) to the solution. Suspension was sonicated for 1 hour at 60° C. Radiochemical yields of absorption and desorption of ^{99m}TcO₄ in nanotubes were determined by loading them on the Teflon filter and washing with ddH2O (Ultra pure, 3 mL), phosphate buffer (PBS 1×, 3 mL), 1 mM solution of EDTA pH=4.3, pH=7.3, and pH=8.3 (each 3 mL). Absorption of GdCl₃ in nanotubes was determined by ICP-MS.

FIG. 8 is a graph depicting the RCY_{Ds} of loading carbon nanotubes with ^{99m}TcO₄ and Gd chloride in 0.1M NaOAc pH=6.1 and 0.1M HEPES pH=8.3, respectively and washing them with 1 mM EDTA.

Loading of Carbon Nanotubes with ⁶⁸Ga or ⁶⁷Ga Chloride and Gd Chloride

US-tubes (100-200 ug) were sonicated for 1 h in 3-ml 0.5M HNO₃ (ultra pure, trace metal free) in water bath at 60° C. and spin down at 10 k rpm for 15 minutes. Ga⁶⁸ chloride was eluted from ⁶⁸Ge/⁶⁸Ga generator (ITGM) using 0.05N HCl (Ultra pure, trace metal free). Nanotubes were resuspended in 0.1M NaOAc buffer pH=6.1, ⁶⁸Ga chloride eluate and GdCl₃ were added (final pH=8.0). Suspension was sonicated for 20 minutes at 60° C. Radiochemical yields of absorption and desorption of ⁶⁸Ga in nanotubes were determined by loading them on the Teflon filter and washing with ddH2O (Ultra pure, 3 mL), phosphate buffer (PBS 1×, 3 mL), 1 mM solution of EDTA pH=4.3, pH=7.3, and pH=8.3 (each 3 mL). Absorption of GdCl₃ inside nanotubes was determined by ICP-MS. RCY and radioactivity of isotope loaded in nanotubes were not back-decayed. Loading of nanotubes with ⁶⁷Ga/Gd was performed according to the protocol described for Ga-68.

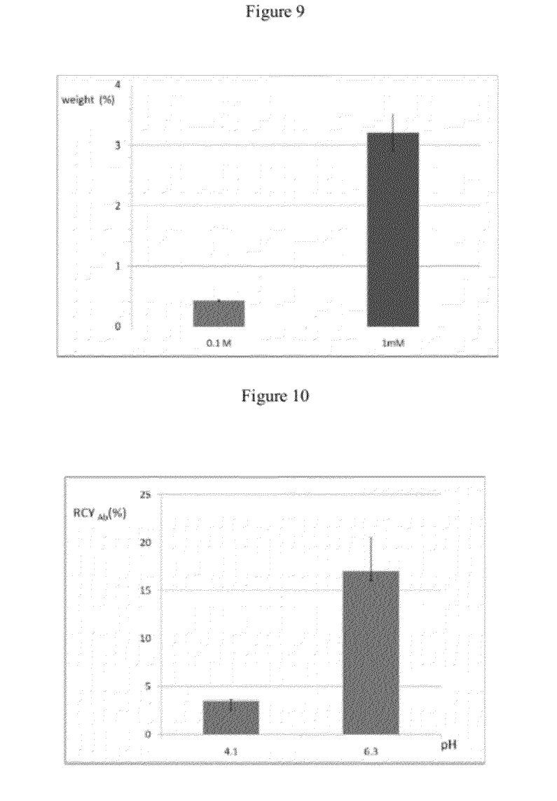

FIG. 9 is a graph depicting the results of loading carbon nanotubes with ⁶⁸Ga and Gd in 0.1M NaOAc and washing it with 0.1M EDTA pH=4.3 and 1 mM EDTA pH=4.3. The graph shows the weight percentages of Gd remained in the carriers after washing them with EDTA as were determined by ICP-MS.

FIG. 10 is a graph depicting the RCY_{Ab} of loading carbon nanotubes with ⁶⁸Ga/Gd in 0.1M NaOAc pH=4.1 and 0.1M NaOAc pH=6.3. Calculated RCY was back-decayed.

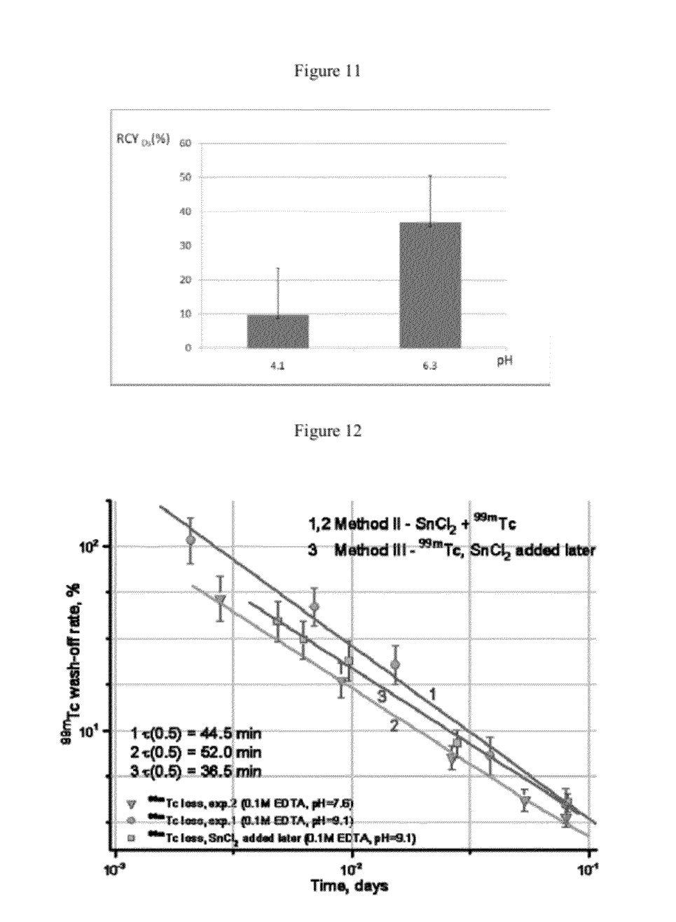

FIG. 11 is a graph depicting the RCY_{Ds} of loading carbon nanotubes with ⁶⁸Ga/Gd in 0.1M NaOAc pH=4.1 and 0.1M NaOAc pH=6.3. Calculated RCY was back-decayed.

Loading of Carbon Nanotubes with Lu¹⁷⁷Chloride

US-tubes (100-200 ug) were sonicated for 1 hour in 3-ml 0.5M HNO₃ (ultra pure, trace metal free) in water bath at 60° C., and spin down at 10 k rpm for 15 minutes. Nanotubes were resuspended in 0.1M NaOAc pH=5.3 and Lu¹⁷⁷ chloride (UM Research Reactor) was added to the solution. Suspension was sonicated for 30 minutes at 60° C. Radiochemical yields of adsorption and desorption of isotope in nanotubes was determined by loading them on the Teflon filter (PALL Life Sciences) and subsequently washing them with ddH2O (Ultra pure, 3 mL), phosphate buffer (PBS 1×, 3 mL), and 0.1M EDTA pH=7.3. RCY and radioactivity of isotope loaded in nanotubes were not back-decayed.

Loading of Carbon Nanotubes with ^{99m}TcO₄

US-tubes (100-200 ug) were sonicated for 1 hour in 3-ml 0.5M HNO₃ (ultra pure, trace metal free) in water bath at 60° C. and spin down at 10 k rpm for 15 minutes. Nanotubes were resuspended in 0.1M NaOAc buffer pH=5.3 and ^{99m}TcO₄ (Triad Isotope Inc.) and SnCl₃ (Sigma Aldrich) were added to the solution. Suspension was sonicated for 1 hour at 60° C. Radiochemical yields of absorption and desorption of ^{99m}TcO₄ in nanotubes were determined by loading them on the Teflon filter and washing with ddH2O (Ultra pure, 3 mL), phosphate buffer (PBS 1×, 3 mL), 1 mM solution of EDTA pH=4.3. RCY and radioactivity of isotope loaded in nanotubes were not back-decayed.

FIG. 12 is a graph depicting the stability of loading the US-tubes with ^{99m}TcO₄ in the presence of SnCl₂ after washing them with PBS buffer (1×, 3 mL) and) and 0.1M EDTA pH=7.6-9.1.

Loading of Carbon Nanotubes with Re Chloride

US-tubes (100-200 ug) were sonicated for 1 hour in 3-ml 0.5M HNO₃ (ultra pure, trace metal free) in water bath at 60° C. and spin down at 10 rpm for 15 minutes. Nanotubes were resuspended in 0.1M NaOAc buffer pH=4.1, ReCl₅ was added and suspension was sonicated for 30 minutes at 60° C. Stability of loading ReCl₅ in nanotubes was determined by loading them on the Teflon filter and washing with ddH2O (Ultra pure, 3 mL), phosphate buffer (PBS 1×, 3 mL), 0.1M solution of EDTA pH=4.3. Absorption of ReCl₅ inside nanotubes was determined by ICP-MS.

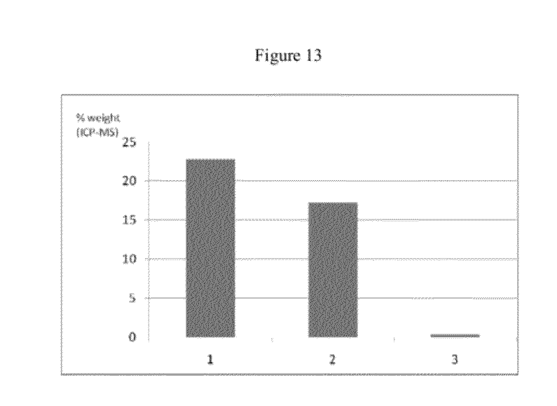

FIG. 13 is a graph depicting the efficacy of loading carbon nanotubes with Re compounds (1) ReCl₅, (2) (NBu)₄ReClO₄— and (3) NH₄ReO₄ as determined by ICP-MS.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A composition comprising:
a Cₙ carrier, wherein Cₙ refers to a fullerene moiety or nanotube comprising n carbon atoms, and
at least one radioisotope.

2. The composition of claim 1 wherein the Cₙ carrier is a buckminsterfullerene, single walled carbon nanotube, or an ultra-short carbon nanotube.

3. The composition of claim 1 wherein the Cₙ carrier is an ultra-short carbon nanotube.

4. The composition of claim 1 wherein the radioisotope comprises at least one radioisotope selected from the group consisting of: an isotope of technetium, an isotope of gallium, an isotope of lutetium, an isotope of indium, an isotope of actinium, and a combination thereof.

5. The composition of claim 1 wherein the radioisotope comprises at least one radioisotope selected from the group consisting of: ^{99m}Tc, ⁶⁸Ga, ¹⁷⁷Lu, ¹¹¹In, ²²⁷Ac, ²²⁵Ac, and a combination thereof.

6. The composition of claim 1 further comprising Gd³⁺.

7. A composition comprising:
a Cₙ carrier, wherein Cₙ refers to a fullerene moiety or nanotube comprising n carbon atoms, and
at least one agent selected from the group consisting of a radioisotope, a metal and a combination thereof.

8. The composition of claim 7 wherein the agent comprises rhenium.

9. The composition of claim 7 wherein the agent comprises at least one radioisotope selected from the group consisting of: an isotope of technetium, an isotope of gallium, an isotope of lutetium, an isotope of indium, an isotope of actinium, and a combination thereof.

10. The composition of claim 9 further comprising Gd³⁺.

11. The composition of claim 7 wherein the agent comprises at least one radioisotope selected from the group consisting of: ^{99m}Tc, ⁶⁸Ga, ¹⁷⁷Lu, ¹¹¹In, ²²⁷Ac, ²²⁵Ac, and a combination thereof.

12. The composition of claim 7 wherein the agent is an imaging agent.

13. The composition of claim 7 wherein the agent is a therapeutic agent.

14. The composition of claim 7 wherein the Cₙ carrier is a buckminsterfullerene, single walled carbon nanotube, or an ultra-short carbon nanotube.

15. The composition of claim 7 wherein the Cₙ carrier is an ultra-short carbon nanotube.

16. A method for imaging comprising:
providing an imaging device;
providing a sample comprising an imaging composition, wherein the imaging composition comprises a Cₙ carrier, wherein Cₙ refers to a fullerene moiety or nanotube comprising n carbon atoms, and at least one radioisotope; and
obtaining an image of at least a portion of the sample using the imaging device.

17. The method of claim 16 wherein the imaging composition further comprises Gd³⁺.

18. The method of claim 16 wherein the Cₙ carrier is an ultra-short carbon nanotube.

19. The method of claim 16 wherein the radioisotope comprises at least one radioisotope selected from the group consisting of: an isotope of technetium, an isotope of gallium, an isotope of lutetium, an isotope of indium, an isotope of actinium, and a combination thereof.

20. The method of claim 16 wherein the radioisotope comprises at least one radioisotope selected from the group consisting of: ^{99m}Tc, ⁶⁸Ga, ¹⁷⁷Lu, ¹¹¹In, ²²⁷Ac, ²²⁵Ac, and a combination thereof.
